(19) United States
(12) Patent Application Publication
(10) Pub. No.: US 2010/0305713 A1
(43) Pub. Date: Dec. 2, 2010

(54) SET FOR PRODUCING AN OFFSET RESURFACING CONDYLE CAP IMPLANT FOR AN ARTIFICIAL HIP JOINT

(75) Inventor: Hans Grundei, Luebeck (DE)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: ESKA IMPLANTS AG, Luebeck (DE)

(21) Appl. No.: 12/675,544

(22) PCT Filed: Aug. 22, 2008
(86) PCT No.: PCT/EP08/60997

§ 371 Date: Feb. 26, 2010

(30) Foreign Application Priority Data
Aug. 30, 2007 (DE) 10 2007 041 794.4

(51) Int. Cl. A61F 2/36

(52) U.S. Cl. 623/23.12

(57) ABSTRACT

A set for producing an offset resurfacing condyle cap implant for an artificial hip joint is described.

The set has
a thin-walled metallic cap (1), to be placed onto the hip joint head that has been milled into shape only in polar area (P) and otherwise has been only decartilaged or freshened, with the walls of the metallic cap being steadily enlarged in the cross-sectional view from of 2 mm to 6 mm in thickness in the area of base edge (2), such that an eccentricity results in its external shape and its interior space (I) has first an essentially cylindrical equatorial area (Z) followed in aforementioned polar area (P) by a truncated cone-shaped, conically narrowing area, with several bore holes (4) in its walls, the axes of which run parallel to main axis (H) of cap (1),
and at least one peg (5) for centering and load-bearing insertion into bore holes (4) in cap (1).

The invention relates to a joint head cap implant for an artificial hip joint, specifically as a replacement surface for the natural articular surface of the hip joint head.

Recently there has been an increase in the use of so-called cap implants, which can be placed over the prepared natural residual joint head of the hip joint and then fixed in this position. Cap implants consist of a cap made to emulate the external form of the natural condyle that can be placed on a (partially) prepared natural residual joint head.

Stable secondary fixation is conditioned on the presence of stable bone material in the residual bone. Thus in DE-C-10218801 it was suggested to join a peg to the joint head cap that would then be placed into a corresponding milled-out recess in the femoral neck. This peg has a surface that is provided with a three-dimensional open-meshed net structure into and through which bone trabeculae of the surrounding bone material grow and provide for stable secondary fixation.

However, there are indications in which the milling-out of the femoral neck, in order to make room for the peg, can be dispensed with.

Reference is made here to so-called Legg-Calvé-Perthes disease, which causes aseptic bone necroses on one or both sides in the area of the femoral head epiphysis. The disease occurs especially in boys from ages four to twelve (Pschyrembel Klinisches Wörterbuch, 259th edition, 2002, p. 1285). Healing without deformation is possible, but a possible trochleiform or fungiform femoral head with flattening of the joint cavity, or more rarely coxa plana or arthrosis deformans may remain.

Another indication, for example, is a cyst in the hip joint head leading to surface defects in the joint head.

In general, necrosis of the joint head may lead to superficial defects that yet do not justify completely resecting the joint head and supplying the patient with a short-stem endoprosthesis (EP 0878176).

Generally speaking—and this has been recognized more and more recently—it is advantageous to wait as long as possible with (partial) resections of bones, in order to be able to resort to multiple stages of endoprosthetic repair, from short-stem to long-stem endoprosthesis, if a corrective intervention later becomes necessary. The use of the last-mentioned endoprosthesis requires the complete resection of the femoral neck.

The peg according to the already cited DE-C-102 18 801 has a bearing and fixing function. However, in this case it is problematic to position the cap in an axially correct manner on the prepared residual joint head. Incorrect positioning can later lead to far-reaching consequences.

DE-U1-20 2006 017 005 discloses a joint head cap implant for an artificial hip joint with a thin-walled metallic cap to be placed onto the natural, merely decartilaged or freshened hip joint head with a guide pin positioned proximally exactly in the middle of the polar area of the cap. The objective of this implant is to be able to position it in an axially correct manner on the natural joint head. The natural joint head is thereby intended to remain largely intact.

EP 1 872 745 A2 discloses an offset resurfacing joint head cap implant for an artificial hip joint that has a metallic cap whose walls are steadily enlarged in the cross-sectional view in the area of the base edge, resulting in an eccentricity in the external shape. A problem with this cap implant is the introduction of stress forces into the femoral neck. Therefore, no information of any kind can be drawn from this document.

There are, however, indications in which, due to the disease picture or wear-and-tear situation, the repair is not optimal with cap implants of the prior art. For example, incorrect positions of the hip joint head might have to be compensated for. In addition, it might be necessary to provide relief from stress forces in the bones of the bony femur that are still intact, even in the case of a largely destroyed polar area or, for example, trocheiform deformation (Perthes, see above). The operator might also have to be able to make adjustments even during the operation.

Accordingly, the task of the present invention is to propose a set for producing an offset resurfacing condyle cap implant for an artificial hip joint that fulfills these aforementioned requirements.

This task is solved by a set with the characteristics of claim 1. Further advantageous embodiments are described in the dependent claims.

Accordingly, the set according to the invention has a thin-walled metallic cap, to be placed onto the hip joint head that has been milled into shape only in the polar area and otherwise has been only decartilaged or freshened, with the walls of the metallic cap being steadily enlarged in the cross-sectional view from 2 mm to 6 mm in thickness in the area of the base edge, such that an eccentricity results in its external shape and its interior space has first—viewed from the base edge—an essentially cylindrical equatorial area followed in the aforementioned polar area by a truncated cone-shaped, conically narrowing area, with several bore holes in its walls, the axes of which run parallel to the main axis of the cap, as well as at least one peg for centering and load-bearing insertion into the bore holes of the cap.

Thus the natural hip joint head is divided into two zones, namely a polar area and an equatorial area. The inventive cap can be used when the equatorial area is still largely intact. It is merely decartilaged or freshened. In contrast, the polar area is so damaged that it must be put into shape with the help of a forming cutter, namely essentially into the shape of a truncated cone that corresponds to the truncated cone-shaped, conically narrowing area in the interior of the cap. The entrance area to the interior of the cap, the equatorial area, has an essentially cylindrical shape. Here the cap implant lies flush with the decartilaged or freshened, but still intact bone of the natural hip joint head. Here the implant acts as a resurfacing implant according to DE 10 2005 011 361 B4.

Due to the special external shape, with its eccentricity because of the steadily increasing thickness of the walls up to a maximum wall thickness and subsequent steady decrease to the minimum wall thickness of the material in the area of the base edge, incorrect positions of the hip joint head can be compensated for.

The required transmission of the arising stress forces into the intact bony tissue of the natural hip joint head or into the femoral neck is accomplished by pegs that are placed into bore holes in the walls of the cap. The insertion of the pegs can be individually adapted to conditions during the operation. Corresponding bony bore holes are brought into the remaining natural joint head, into which the pegs are inserted in the implantation.

Using a “trial cap” that has the same external contour as the actual cap implant, the operator can rotate it on the (residual) joint head to determine the position of the metallic cap to be placed then onto the hip joint head. Because of the eccentricity of the external shape of the cap, the operator can use the “trial cap” to simulate a compensation for the incorrect positioning of the joint head. The metallic implant is then placed into this same position which was found to be optimal with the “trial cap.”

For an intimate connection between the peg and the bone material enveloping it, it can advantageously be provided that the pegs are furnished with an osteoinductive coating. For example, this might be hydroxylapatite or the like. This aims to create a permanent intimate bond between the peg material and the bone material.

The metallic cap can be cemented onto the hip joint head. But it is also possible to implant a cap without the use of cement. In this case, the interior space of the cap is provided at least partially with an open-meshed three-dimensional net structure, into and through which bone trabeculae can grow for a stable secondary fixation.

The invention is described in greater detail using an embodiment according to the drawing Figures.

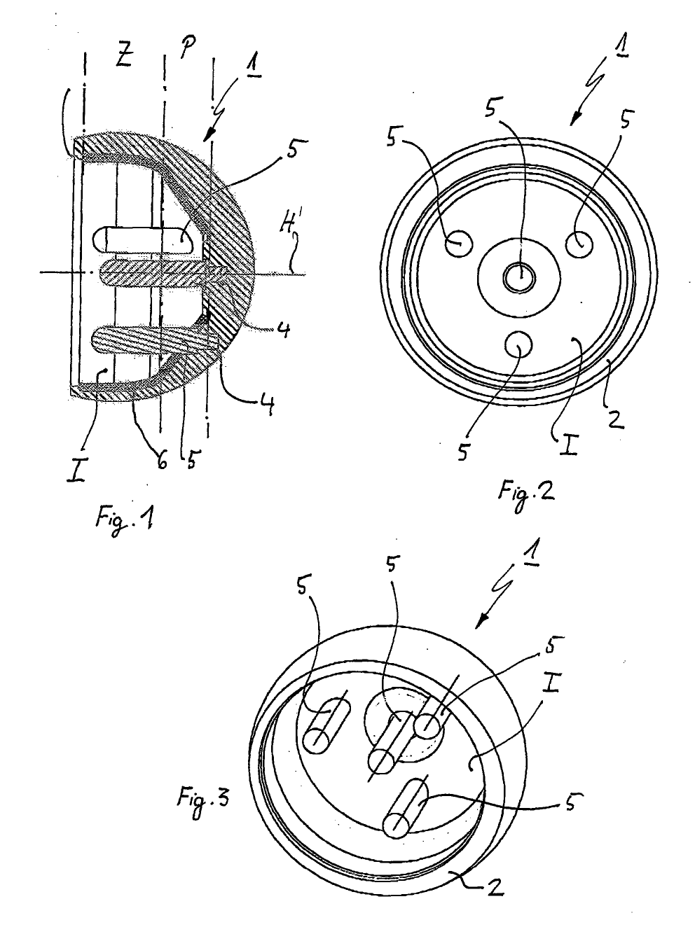

FIG. 1 shows a cross-sectional view of a cap implant,

FIG. 2 shows a view of the interior of the cap implant of FIG. 1, and

FIG. 3 shows an isometric view of the cap implant.

The cap implant produced from the set has a thin-walled metallic dome-shaped cap 1 with a main axis H. The area near the entrance to interior I of cap 1 is designated equatorial area Z. The interior in equatorial area Z of cap 1 runs essentially cylindrically. This area acts as a pure resurfacing implant with its flush position to the equatorial area of the natural hip joint head, once the latter is decartilaged or freshened.

Polar area P of cap 1, on the other hand, has a truncated cone shape. The natural hip joint bone must be prepared accordingly with a forming cutter. Since cap 1 is provided for indications in which the natural polar area of the hip joint head is largely destroyed or else deformed, care must be taken to ensure that the stress forces especially from the polar area are transmitted into the bone of the residual joint head or into the femoral neck. This is achieved through pegs 5 that are inserted into bore holes 4 in the walls of cap 1. Their axes are essentially parallel in axis to main axis H of cap 1.

Interior I of cap 1 is provided in the depicted embodiment with an open-meshed three-dimensional net structure 6, into and through which bone trabeculae grow and thus provide for a stable long-term fixation. Thus, depicted cap 1 can be implanted without the use of cement.

In the depictions of FIGS. 2 and 3, the open-meshed three-dimensional net structure has been omitted. Pegs 5 in FIG. 3 are also only shown schematically, especially the distal end of the peg.

The formulated goals are achieved in the present case by means of the following components:

1. Pegs to transmit the stress forces into intact bone material.
2. Pure resurfacing components in the equatorial area of the cap.
3. The truncated cone-shaped formation of the interior of cap 1 in the polar area.
4. The external eccentricity of cap 1.

1. A set for producing an offset resurfacing condyle cap implant for an artificial hip joint, having
a thin-walled metallic cap (1), to be placed onto the hip joint head that has been milled into shape only in polar area (P) and otherwise has been only decartilaged or freshened, with the walls of the metallic cap being steadily enlarged in the cross-sectional view from of 2 mm to 6 mm in thickness in the area of base edge (2), such that an eccentricity results in its external shape and its interior space (I) has first an essentially cylindrical equatorial area (Z) followed in aforementioned polar area (P) by a truncated cone-shaped, conically narrowing area, with several bore holes (4) in its walls, the axes of which run parallel to main axis (H) of cap (1),
and at least one peg (5) for centering and load-bearing insertion into bore holes (4) in cap (1).

2. The set of claim 1, in which the surface of pegs (5) is provided with an osteoinductive coating.

3. The set of claim 1 or 2, in which interior space (I) of cap (1) is provided at least partially with an open-meshed three-dimensional net structure (6).
